(19) United States
(12) Patent Application Publication
(10) Pub. No.: US 2007/0110816 A1
(43) Pub. Date: May 17, 2007

(54) METHOD OF COATING NANOPARTICLES

(76) Inventors: Shin-ae Jun, Yongin-si (KR); Eun Joo Jang, Yongin-si (KR); Jung Eun Lim, Yongin-si (KR)

Correspondence Address: CANTOR COLBURN, LLP, 55 GRIFFIN ROAD SOUTH, BLOOMFIELD, CT, 06002, US

(21) Appl. No.: 11/558,937

(22) Filed: Nov. 13, 2006

(30) Foreign Application Priority Data
Nov. 11, 2005 (KR) 1020050108122

(51) Int. Cl. A61K 9/28; A61K 9/50

(52) U.S. Cl. 424/490; 977/906; 427/2.14

(57) ABSTRACT

Disclosed herein is a method of coating nanoparticles with a metal oxide. The method includes substituting surfaces of hydrophobic nanoparticles with an organic substance having a hydrophilic group effective to render the nanoparticles hydrophilic; and injecting the hydrophilic nanoparticles and a precursor of the metal oxide into an organic solvent including an amphiphilic surfactant to coat the nanoparticles with a metal oxide.

This application claims priority to Korean Patent Application No. 10-2005-0108122, filed on Nov. 11, 2005, in the Korean Intellectual Property Office, and all the benefits accruing therefrom under 35 U.S.C. § 119, the contents of which are incorporated herein by reference in its entirety.

### BACKGROUND OF THE INVENTION

1. Field of the Invention

The present invention relates to a method of coating nanoparticles using a metal oxide. More particularly, the present invention relates to a method of uniformly coating nanoparticles using a metal oxide by uniformly transferring nanoparticles surface-treated using a hydrophilic surfactant into a micelle structure of an amphiphilic surfactant formed in an organic solvent, and by injecting a precursor of the metal oxide into the micelle structure.

2. Description of the Related Art

Quantum dots, which are compound semiconductor nanoparticles, are representative examples of nanoparticles. The quantum dots formed of semiconductor material have a size on the scale of nanometers and exhibit a quantum confinement effect. When excited by light emitted from an excitation source, quantum dots emit energy according to an energy band gap thereof. Therefore, quantum dots can be used as a light-emitting materials emitting visible and infrared light. On the other hand, the quantum dots can be used for a light receiving material since the quantum dots generate a current when visible or infrared light is absorbed. Thus, the quantum dots are considered to be next generation of electronics materials.

Since quantum dots made by wet chemical methods are dispersed throughout a solvent in a colloidal state, a coating process is performed on the quantum dots for various reasons. Examples of coating methods of nanoparticles include epitaxial growth of a different material on the surfaces of the quantum dots while maintaining appropriate crystal faces, surface-initiated polymerization that forms a precursor of a coating material on the surfaces of the nanoparticles and transforms the precursor into a coating material through polymerization, and amorphous coating such as sol-gel coating or concentrated liquid coating.

In an existing method of coating nanoparticles using water-in-oil inverse micelles (one type of sol-gel coating method), nanoparticles capped with a hydrophobic surfactant are injected into a hydrophobic solvent to disperse the nanoparticles throughout the hydrophobic solvent. Since the cores of the micelles, which are formed by an amphiphilic surfactant in the hydrophobic solvent, are hydrophilic, the nanoparticles coated with the hydrophobic surfactant are not readily transferred to the cores of the micelles. Also, some nanoparticles that are transferred to the cores of the micelles agglomerate at the hydrophilic cores of the micelles. If a precursor of a metal oxide is injected into the hydrophobic solvent under these conditions, the metal oxide may be formed on the surfaces of the agglomerated nanoparticles or formed into discrete particles that do not contain any nanoparticles. For example, when CeSeS nanoparticles combined with an oleic acid are coated with silica according to an existing method, the CeSeS nanoparticles agglomerate in the hydrophilic cores of the silica molecules because the surfaces of the CeSeS nanoparticles are hydrophobic. Therefore, it is difficult to coat the nanoparticles uniformly.

FIG. 1 is an electron microscope image of CdSeS nanoparticles coated with silica using an existing method of coating nanoparticles. As shown in the figure, the CdSeS nanoparticles (black) are not uniformly coated with the silica (gray), and the number of CdSeS nanoparticles present in one given agglomerate coated with the silica varies largely from agglomerate to agglomerate. Therefore, there is a need for an improved method of uniformly coating nanoparticles.

### BRIEF SUMMARY OF THE INVENTION

The present invention provides a method of coating nanoparticles using a metal oxide, wherein the method includes altering the surfaces of the nanoparticles to become hydrophilic.

According to an exemplary embodiment of the present invention, a method of coating nanoparticles with a metal oxide includes substituting surfaces of hydrophobic nanoparticles with an organic substance having a hydrophilic group effective to render the nanoparticles hydrophilic; and injecting the hydrophilic nanoparticles and a precursor of the metal oxide into an organic solvent including an amphiphilic surfactant to coat the nanoparticles with the metal oxide.

The substituting of the surfaces of the hydrophobic nanoparticles may include substituting the surfaces of the hydrophobic nanoparticles with a surfactant having a hydrophilic group to render the nanoparticles hydrophilic, and dispersing the hydrophilic nanoparticles into a hydrophilic solvent.

The hydrophilic surfactant may include one of pyridine, dithiol, mercaptoalkyalchol, mercaptoalkylamine, mercaptoalkylsilane, aminoalkylsilane, and a diamine.

The hydrophilic solvent may be a hydrophilic organic solvent selected from the group consisting of an primary alcohol, a secondary alcohol, a diol, a polyol, a cyclic amine, a cyclic ether, and a ketone.

The nanoparticles may be selected from the group consisting of a group II-VI compound, a group III-V compound, a group IV-VI compound, a group IV compound, and a combination comprising at least one of the foregoing. Furthermore, the nanoparticles can have a core-shell or multishell structure.

Any of the group II-VI compound, group III-V compound, or group IV-VI compound may be a multinary compound (e.g., a binary compound, ternary compound, quaternary compound, or a more complex compound). The group IV compound may be a single-element substance or a binary compound.

Each component of the multinary compounds may be included in each of the nanoparticles in a uniform concentration or in a gradient (i.e., at different concentrations from one portion of the nanoparticle to another).

The group II-VI compound may be selected from the group consisting of CdSe, CdTe, ZnS, ZnSe, ZnTe, ZnO, HgS, HgSe, HgTe, CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, and HgZnSe, HgZnTe, CdZnSeS, CdZnSeTe, CdZnSTe, CdHgSeS, CdHgSeTe, CdHgSTe, HgZnSeS, HgZnSeTe, and HgZnSTe.

The group III-V compound may be selected from the group consisting of GaN, GaP, GaAs, GaSb, AlN, AlP, AlAs, AlSb, InN, InP, InAs, InSb, GaNP, GaNAs, GaNSb, GaPAs, GaPSb, AlNP, AlNAs, AlNSb, AlPAs, AlPSb, InNP, InNAs, InNSb, InPAs, InPSb, GaAlNP, GaAlNAs, GaAlNSb, GaAlPAs, GaAlPSb, GaInNP, GaInNAs, GaInNSb, GaInPAs, GaInPSb, InAlNP, InAlNAs, InAlNSb, InAlPAs, and InAlPSb.

The group IV-VI compound may be selected from the group consisting of SnS, SnSe, SnTe, PbS, PbSe, PbTe, SnSeS, SnSeTe, SnSTe, PbSeS, PbSeTe, PbSTe, SnPbS, SnPbSe, SnPbTe, SnPbSSe, SnPbSeTe, and SnPbSTe.

The group IV compound may be selected from the group consisting of Si, Ge, SiC, and SiGe.

The nanoparticles may be selected from the group consisting of Pd, Pt, Ni, Co, Rh, Ir, Fe, Ru, Au, Ag, Cu, and combinations thereof.

The metal oxide may be selected from the group consisting of SiO₂, TiO₂, SnO₂, ZnO, ZnS, In₂O₃—SnO₂, Al₂O₃, HfO₂, BaTiO₃, CeO₂, ZrO₂, Ta₂O₅ and combinations thereof.

The precursor of the metal oxide may be selected from the group consisting of triethoxysilane, trimethoxysilane, tributhoxysilane, sodium silicate, titanium isopropoxide, titanium butoxide, tin butoxide, and sodium stannate.

### BRIEF DESCRIPTION OF THE DRAWINGS

The above and other features and advantages of the present invention will become more apparent from the following detailed description taken in conjunction with the attached drawings, in which:

FIG. 1 is an electron microscope image of nanoparticles coated with a metal oxide according to an existing method;

FIGS. 2A through 2E are schematic illustrations of an exemplary embodiment of a method of coating nanoparticles according to the present invention;

FIG. 3 is an emission spectrum of optically excited CdSeS nanoparticles;

FIG. 4A is an emission spectrum of optically excited CdSeS nanoparticles coated with SiO₂;

FIG. 4B is an electron microscope image of CdSeS nanoparticles coated with SiO₂;

FIG. 5A is another electron microscope image of CdSeS nanoparticles coated with SiO₂;

FIG. 5B is an electron microscope image of CdSeS nanoparticles coated with SnO; and

FIG. 5C is an electron microscope image of Pd nanoparticles coated with SiO₂.

### DETAILED DESCRIPTION OF THE INVENTION

The present invention will now be described more fully hereinafter with reference to the accompanying drawings, in which exemplary embodiments of the present invention are shown. This invention may, however, be embodied in many different forms and should not be construed as limited to the exemplary embodiments set forth herein. Rather, these exemplary embodiments are provided so that this disclosure will be thorough and complete, and will fully convey the scope of the invention to those skilled in the art. Like reference numerals refer to like elements throughout.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” or “includes” and/or “including” when used in this specification, specify the presence of stated features, regions, steps, elements, and/or components, but do not preclude the presence or addition of one or more other features, regions, steps, elements, components, and/or groups thereof.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

The nanoparticles for use in the methods disclosed herein are generally made by a wet chemical process. In the wet chemical process, a surfactant of a selected type is added to a proper solvent in a specific concentration under an inert atmosphere (e.g., nitrogen or argon gas atmosphere), and the solution is maintained at a specified reaction temperature to grow crystals. Next, a precursor of the nanoparticles is injected into the solution, and the solution is left for a specific time to grow nanoparticles up to a desired size. After that, the solution is cooled, and nanoparticles are separated from the solution. The nanoparticles made through the above-described wet chemical process have hydrophobic surfaces. Therefore, when the nanoparticles are added to a hydrophilic medium, the nanoparticles are generally non-uniformly dispersed and agglomerate. To prevent this, the nanoparticles are treated to make their surfaces hydrophilic.

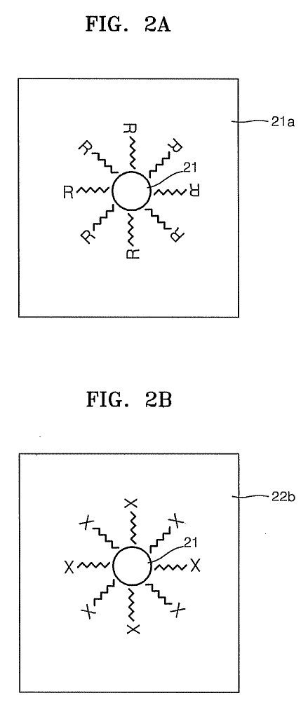

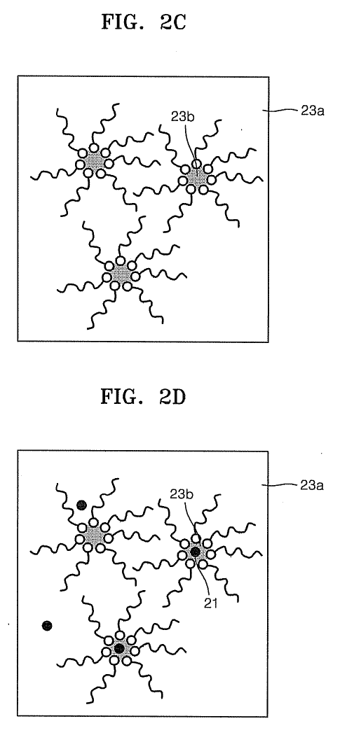

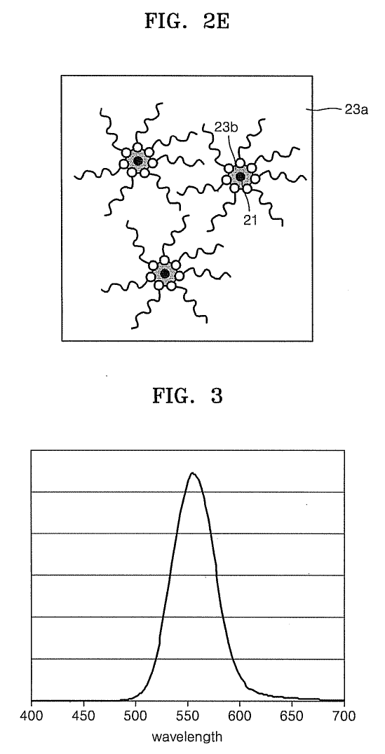

FIGS. 2A through 2E schematically illustrate an exemplary embodiment of a method of coating nanoparticles according to the present invention.

Referring now to FIG. 2A, nanoparticles 21 made by a wet chemical process are present in a hydrophobic solvent 21a. The nanoparticles 21 have hydrophobic surfaces, generally designated by “R”.

Referring to FIG. 2B, to change the surfaces (R) of the nanoparticles 21 from hydrophobic to hydrophilic, the nanoparticles 21 can be injected into a dispersion solution 22a containing a hydrophilic surfactant. The dispersion is stirred until the hydrophobic surfaces (R) are exchanged with hydrophilic surfaces, which are generally designated by “X”. If desired, the surface exchange can be repeated after separation. For example, hydrophilic surfactants such as pyridine, dithiol, mercaptoalkyalchol, mercaptoalkylamine, mercaptoalkylsilane, aminoalkylsilane, or diamine can be used for the surface exchange. Further, various hydrophilic solvents can be used for the dispersion, including for example, primary and secondary alcohols such as methanol, ethanol, propanol, butanol, isopropanol, isobutanol, or tert-butanol; diols such as ethylene glycol, propylene glycol, or butylene glycol; polyols such as polyethylene glycol; cyclic amines such as pyridine or imidazole; cyclic ethers such as tetrahydrofuran; or ketones such as acetone. In this manner, the nanoparticles 21 can be uniformly dispersed throughout the hydrophilic dispersion.

Referring to FIG. 2C, wherein amphiphilic surfactant molecules form micelles in an organic solvent 23a. Known amphiphilic surfactants such as BRIJ, IGEPAL, TX-100, block copolymers (Pluronic P123, F127, and the like) can be used. A non-polar solvent can be used for the organic solvent. When an amphiphilic surfactant is injected into the organic solvent 23a, hydrophilic parts of amphiphilic surfactant molecules gather closely, forming micelle cores 23b. Referring to FIG. 2D, the nanoparticles 21 with hydrophilic surfaces (X) are injected into the organic solvent 23a. Since the nanoparticles 21 now have hydrophilic surfaces (X), the nanoparticles 21 are readily transferred into the micelle cores 23b formed by the amphiphilic surfactant molecules.

Referring to FIG. 2E, a precursor of a metal oxide, water, and an acid or base catalyst for polymerization are injected into the organic solvent 23a. Generally, the precursor of the metal oxide is hydrophilic. Therefore, the precursor moves to the cores 23b of the micelles formed by the amphiphilic surfactant and forms the metal oxide on the surfaces of the nanoparticles 21 in the cores 23b of the micelle. The metal oxide formed on the surfaces of the nanoparticles 21 increases the stability of the surfaces of the nanoparticles 21. In addition, when the nanoparticles 21 are used as a light emitting unit, the metal oxide may increase the light emitting efficiency and lifetime of the light emitting unit.

In an exemplary embodiment, the nanoparticles may be one of a group II-VI compound, a group II-V compound, a group IV-VI compound, a group IV compound, a noble metal, a transition metal, or a combination comprising at least one of the foregoing. The nanoparticles may have a core-shell or multishell structures.

Any of the group II-VI compound, a group III-V compound, a group IV-VI compound, a group IV compound may be a multinary compound. As used herein, the term “multinary compound” is used for convenience, and is intended to encompass a binary compound, a ternary compound, a quaternary compound, or even more complex compounds.

In exemplary embodiments, when a group II-VI compound is used, it is a binary compound, a ternary compound, or a quaternary compound; the group III-V compound is a binary compound, a ternary compound, or a quaternary compound; the group IV-VI compound is a binary compound, a ternary compound, or a quaternary compound; and the group IV compound is a single-element substance or a binary compound.

Each component in the multinary compounds may be present in each of the nanoparticles in a uniform concentration or in a gradient (i.e., at different concentrations from one portion of the nanoparticle to another).

Exemplary group II-VI compounds include CdSe, CdTe, ZnS, ZnSe, ZnTe, ZnO, HgS, HgSe, HgTe, CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, and HgZnSe, HgZnTe, CdZnSeS, CdZnSeTe, CdZnSTe, CdHgSeS, CdHgSeTe, CdHgSTe, HgZnSeS, HgZnSeTe, and HgZnSTe. Exemplary group III-V compounds include GaN, GaP, GaAs, GaSb, AlN, AlP, AlAs, AlSb, InN, InP, InAs, InSb, GaNP, GaNAs, GaNSb, GaPAs, GaPSb, AlNP, AlNAs, AlNSb, AlPAs, AlPSb, InNP, InNAs, InNSb, InPAs, InPSb, GaAlNP, GaAlNAs, GaAlNSb, GaAlPAs, GaAlPSb, GaInNP, GaInNAs, GaInNSb, GaInPAs, GaInPSb, InAlNP, InAlNAs, InAlNSb, InAIPAs, and InAIPSb. Exemplary group IV-VI compounds include SnS, SnSe, SnTe, PbS, PbSe, PbTe, SnSeS, SnSeTe, SnSTe, PbSeS, PbSeTe, PbSTe, SnPbS, SnPbSe, SnPbTe, SnPbSSe, SnPbSeTe, and SnPbSTe. Exemplary group IV compounds include Si, Ge, SiC, and SiGe. Exemplary noble metals and transition metals include Pd, Pt, Ni, Co, Rh, Ir, Fe, Ru, Au, Ag, Cu, or a combination comprising at least one of the foregoing.

The metal oxide to be coated on the surface of the nanoparticles may comprise SiO₂, TiO₂, SnO₂, ZnO, ZnS, In₂O₃—SnO₂, Al₂O₃, HfO₂, BaTiO₃, CeO₂, ZrO₂, Ta₂O₅, or a combination comprising at least one of the foregoing.

The thickness of the metal oxide formed on the nanoparticles is not limited. In an exemplary embodiment, the thickness of the metal oxide is about 1 nanometer (nm) to about 100 nm.

The present invention will be described in greater detail with reference to the following examples. The following examples are for illustrative purposes and are not intended to limit the scope of the present invention.

### EXAMPLE 1

### Process for Manufacturing Cyclohexane, Pyridine, and Butanol Solutions of CdSeS Nanoparticles

16 grams (g) trioctylamine (TOA), 0.5 g oleic acid, and 0.4 millimoles (mmol) cadmium oxide were poured into a 125 milliliter (ml) flask in which a reflux condenser was installed; and the mixture was stirred at a temperature of 300 degrees Celsius (° C.) to prepare a clean reaction mixture. Separately, Se powder was dissolved in trioctyphosphine (TOP) to prepare an Se-TOP complex solution having a concentration of about 0.25 moles of solute per liter of solvent (M), and S powder was dissolved in TOP to prepare an S-TOP complex solution having a concentration of about 1.0 M. A mixture of 0.9 ml of the S-TOP complex solution and 0.1 ml of the Se-TOP complex solution was rapidly injected into the reaction mixture including cadmium, and the reaction mixture was further stirred for about 4 minutes. After reaction, the reaction mixture was rapidly cooled. Next, ethanol (non-solvent) was added to the reaction mixture, and the reaction mixture was centrifuged. Then, the supernatant of the centrifuged reaction mixture solution was decanted, and the remaining precipitate was dispersed in cyclohexane to obtain a cyclohexane solution having about 1 weight percent (wt %) CdSeS nanoparticles.

Ethanol (non-solvent) was added to the cyclohexane solution with CdSeS nanoparticles, and the mixture was centrifuged. The precipitate of the centrifuged mixture was dispersed in pyridine and stirred for about 30 minutes. Then, hexane (non-solvent) was added to the agitated mixture of the precipitate and the pyridine, and the mixture was centrifuged again. Then, the resultant precipitate was dispersed independently in pyridine and in butanol to prepare 1 wt % pyridine and butanol solution of CdSeS nanoparticles, respectively. The CdSeS nanoparticles dispersed in the pyridine and the butanol were able to be used as quantum dots emitting light having a wavelength of 522 nm. An emission spectrum of the CdSeS nanoparticles excited at 365 nm is shown in FIG. 3.

### EXAMPLE 2

### Process for Coating SiO₂ on CdSeS Nanoparticles by Using Pyridine Solution

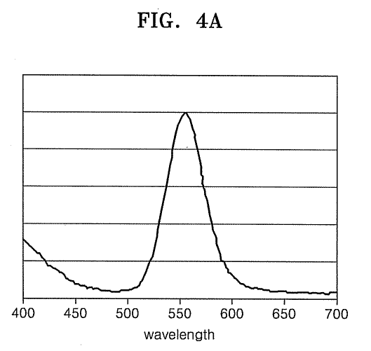

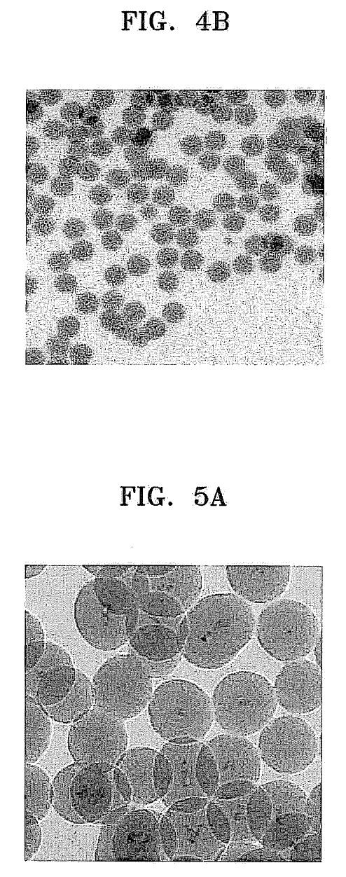

0.1 g IGEPAL CO-520 was added to 2 ml cyclohexane, and the mixture was stirred at room temperature for about 30 minutes to obtain an IGEPAL CO-520/cyclohexane solution. 40 microliters (μl) of the 1 wt % pyridine solution of CdSeS nanoparticles manufactured according to Example 1 were added to the IGEPAL CO-520/cyclohexane solution, and then the mixed solution was stirred at room temperature for about 30 minutes. Next, a 50 μl NH₄OH solution was added to the mixture and then the solution was stirred again at room temperature for about 1 hour. 10-μl tetraethylorthosilicate (TEOS) was added to the solution, and the solution was stirred at room temperature for about 24 hours. After stirring, methanol was added to the solution, and the solution was centrifuged. The precipitate of the centrifuged solution was dispersed in ethanol. FIG. 4A is an emission spectrum excited at 365 nm of the SiO₂-coated CdSeS nanoparticles, and FIG. 4B is an electron microscope image of the SiO₂-coated CdSeS nanoparticles. Referring to FIG. 4A, SiO₂-coated CdSeS nanoparticles were able to emit light having a wavelength of 522 nm to the same extent as the CdSeS nanoparticles of Example 1.

### EXAMPLE 3

### Process for Coating SiO₂ on CdSeS Nanoparticles by Using Butanol Solution

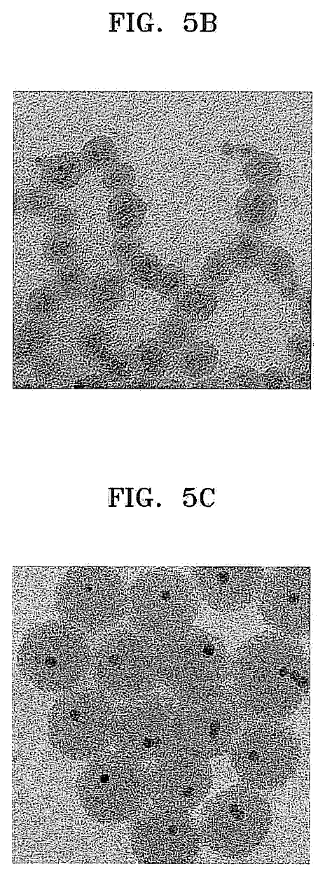

0.1 g IGEPAL CO-520 was added to 2 ml cyclohexane, and the mixture was stirred at room temperature for 30 minutes to obtain an IGEPAL Co-520/cyclohexane solution. 40 μl of the 1 wt % butanol solution of CdSeS nanoparticies manufactured according to Example 1 were added to the IGEPAL Co-520/cyclohexane solution, and the mixture was stirred at room temperature for 30 minutes. Next, a 50 82 l NH₄OH solution was added to the mixture, and the mixture was further stirred at room temperature for 1 hour. Next, 10 μl TEOS was added to the mixture, and the mixture was stirred at room temperature for 24 hours. After that, methanol was added to the mixture, and the mixture was centrifuged. The precipitate of the centrifuged mixture was dispersed in ethanol. FIG. 5A is an electron microscope image of the SiO₂-coated CdSeS nanoparticles.

### EXAMPLE 4

### Process for Coating SnO on CdSeS Nanoparticles

0.1 g IGEPAL CO-520 was added to 2 ml cyclohexane, and the mixture was stirred at room temperature for 30 minutes to obtain an IGEPAL Co-520/cyclohexane solution. 40 μl of the 1 wt % pyridine solution of CdSeS nanoparticles manufactured according to Example 1 were added to the IGEPAL Co-520/cyclohexane solution, and the mixture was stirred at room temperature for 30 minutes. Next, a 40 μl NH₄OH solution was added to the mixture, and the mixture was further stirred at room temperature for 1 hour. Next, a 10 μl sodium stannate aqueous solution was added to the mixture, and the mixture was stirred at room temperature for 24 hours. After that, methanol was added to the mixture, and the mixture was centrifuged. The precipitate of the centrifuged mixture was dispersed in ethanol. FIG. 5B is an electron microscope image of the SnO coated CdSeS nanoparticles.

### EXAMPLE 5

### Process for Coating SiO₂ on Pd Nanoparticles

1 ml TOP, 9 ml olelyamine, and 0.1 g Pd(acetylacetonate) were poured into a 125-ml flask in which a reflux condenser was installed, and the mixture was stirred and gradually heated to a temperature of 260° C. for reaction. After that the mixture was maintained at the reaction temperature for 30 minutes with stirring. After reaction, the reaction mixture was rapidly cooled. Next, ethanol (non-solvent) was added to the reaction mixture, and the reaction mixture was centrifuged. The supernatant of the centrifuged mixture was decanted, and the remaining precipitate was dispersed in hexane to prepare a 1 wt % hexane solution of Pd nanoparticles.

Ethanol (non-solvent) was added to the hexane solution having the Pd nanoparticles, and the solution was centrifuged. The precipitate of the centrifuged solution was dispersed and stirred in pyridine solution for about 30 minutes. After that, hexane (non-solvent) was added to the solution, and the solution was centrifuged. Next, the precipitate of the centrifuged solution was dispersed in pyridine solution to prepare 1 wt % solution.

0.1 g IGEPAL CO-520 was added to 2 ml cyclohexane, and the mixture was stirred at room temperature for about 30 minutes to obtain an IGEPAL CO-520/cyclohexane solution. 40 μl of the 1 wt % pyridine solution of Pd nanoparticles made above were added to the IGEPAL CO-520/cyclohexane solution, and then the mixed solution was stirred at room temperature for about 30 minutes. Next, a 50 μl NH₄OH solution was added and further stirred at room temperature for about 1 hour. Then, the solution was mixed with 10 μl TEOS and stirred at room temperature for about 24 hours. After stirring, the solution was mixed with methanol and centrifuged. The precipitate of the centrifuged solution was dispersed in ethanol. FIG. 5C is an electron microscope image of the SiO₂ coated Pd nanoparticles.

### Comparative Example 1

### Conventional Process for Coating CdSeS Nanoparticles with SiO₂

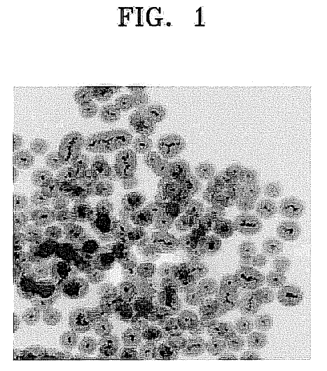

0.1 g IGEPAL CO-520 was added to 2 ml cyclohexane, and the mixture was agitated at room temperature for about 30 minutes to obtain an IGEPAL CO-520/cyclohexane solution. 40 μl of 1 wt % cyclohexane solution of CdSeS nanoparticles having hydrophobic surfaces was added to the IGEPAL CO-520/cyclohexane solution, and then the mixed solution was stirred at room temperature for about 30 minutes. Next, a 50 μl NH₄OH solution was added to the solution and stirred again at room temperature for about 1 hour. 10-μl TEOS were added to the solution, and the solution was further stirred at room temperature for about 24 hours. After stirring, methanol was added to the solution, and the solution was centrifuged. The precipitate of the centrifuged solution was dispersed in ethanol. FIG. 1 is an electron microscope image of the SiO₂ coated CdSeS nanoparticles thus prepared.

Electron microscope images of the nanoparticies manufactured by the coating method according to embodiments of the present invention are shown in FIGS. 4B, 5A, 5B, and 5C, and an electron microscope image of the nanoparticles manufactured by an existing method is shown in FIG. 1. As shown in FIGS. 4B, 5A, 5B, and 5C, according to exemplary embodiments of the present invention, a metal oxide was uniformly formed on each nanoparticle. However, the nanoparticles manufactured by the known method are not uniformly coated with a metal oxide. Instead, the metal oxide is non-uniformly formed on agglomerates of the nanoparticles.

According to the present invention, when nanoparticles are coated with a metal oxide using micelles, the surfaces of the hydrophobic nanoparticles are first substituted with a hydrophilic surfactant. Therefore, the now hydrophilic nanoparticles can be uniformly injected into the hydrophilic cores of the micelles, and thus the nanoparticles can be uniformly coated with the metal oxide.

Although the present invention has been described with reference to the foregoing exemplary embodiments, these exemplary embodiments do not serve to limit the scope of the present invention. Accordingly, those skilled in the art to which the present invention pertains will appreciate that various changes, additions, and substitutions are possible, without departing from the spirit and scope of the accompanying claims.

What is claimed is:

1. A method of coating nanoparticles with a metal oxide, comprising:
substituting surfaces of hydrophobic nanoparticles with an organic substance having a hydrophilic group to render the nanoparticles hydrophilic; and
injecting the hydrophilic nanoparticles and a precursor of the metal oxide into an organic solvent including an amphiphilic surfactant to coat the nanoparticles with the metal oxide.

2. The method of claim 1, wherein the substituting of the surfaces of the hydrophobic nanoparticles comprises:
substituting the surfaces of the hydrophobic nanoparticles with a hydrophilic surfactant to render the nanoparticles hydrophilic; and
dispersing the hydrophilic nanoparticles in a hydrophilic solvent.

3. The method of claim 2, wherein the hydrophilic surfactant comprises one of pyridine, dithiol, mercaptoalkyalchol, mercaptoalkylamine, mercaptoalkylsilane, aminoalkylsilane, and diamine.

4. The method of claim 2, wherein the hydrophilic solvent is selected from the group consisting of a primary alcohol, a secondary alcohol, a diol, a polyol, a cyclic amine, a cyclic ether, and a ketone.

5. The method of claim 2, wherein the hydrophilic solvent is a hydrophilic solvent selected from the group consisting of methanol, ethanol, propanol, butanol, isopropanol, isobutanol, tert-butanol, ethylene glycol, propylene glycol, butylene glycol, polyethylene glycol, pyridine, imidazole, tetrahydrofuran, and acetone.

6. The method of claim 1, wherein the nanoparticles are selected from the group consisting of a group II-VI compound, a group III-V compound, a group IV-VI compound, a group IV compound, a noble metal, a transition metal and a combination comprising at least one of the foregoing.

7. The method of claim 6, wherein the group II-VI compound, the group III-V compound, or the group IV-VI compound comprises a multinary compound.

8. The method of claim 7, wherein the group II-VI compound is a binary compound, a ternary compound, or a quaternary compound.

9. The method of claim 7, wherein the group III-V compound is a binary compound, a ternary compound, or a quaternary compound.

10. The method of claim 7, wherein the group IV-VI compound is a binary compound, a ternary compound, or a quaternary compound.

11. The method of claim 6, wherein the group IV compound is a single-element composition or a binary compound.

12. The method of claim 7, wherein each component of the multinary compound is included in each of the nanoparticles in a uniform concentration or in a gradient.

13. The method of claim 6, wherein the group II-VI compound is selected from the group consisting of CdSe, CdTe, ZnS, ZnSe, ZnTe, ZnO, HgS, HgSe, HgTe, CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, and HgZnSe, HgZnTe, CdZnSeS, CdZnSeTe, CdZnSTe, CdHgSeS, CdHgSeTe, CdHgSTe, HgZnSeS, HgZnSeTe, and HgZnSTe.

14. The method of claim 6, wherein the group III-V compound is selected from the group consisting of GaN, GaP, GaAs, GaSb, AIN, AIP, AIAs, AISb, InN, InP, InAs, InSb, GaNP, GaNAs, GaNSb, GaPAs, GaPSb, AINP, AINAs, AINSb, AIPAs, AIPSb, InNP, InNAs, InNSb, InPAs, InPSb, GaAINP, GaAINAs, GaAINSb, GaAIPAs, GaAIPSb, GaInNP, GaInNAs, GaInNSb, GaInPAs, GalnPSb, InAINP, InAINAs, InAINSb, InAIPAs, and InAIPSb.

15. The method of claim 6, wherein the group IV-VI compound is selected from the group consisting of SnS, SnSe, SnTe, PbS, PbSe, PbTe, SnSeS, SnSeTe, SnSTe, PbSeS, PbSeTe, PbSTe, SnPbS, SnPbSe, SnPbTe, SnPbSSe, SnPbSeTe, and SnPbSTe.

16. The method of claim 6, wherein the group IV compound is selected from the group consisting of Si, Ge, SiC, and SiGe.

17. The method of claim 6, wherein the noble metal or the transition metal is Pd, Pt, Ni, Co, Rh, Ir, Fe, Ru, Au, Ag, Cu, or a combination comprising at least one of the foregoing.

18. The method of Claim, 1, wherein the metal oxide is SiO₂, TiO₂, SnO₂, ZnO, ZnS, In₂O₃-SnO₂, Al₂O₃, HfO₂, BaTiO₃, CeO₂, ZrO₂, Ta₂O₅, or comprising at least one of the foregoing.

19. The method of claim 1, wherein the precursor of the metal oxide is selected from the group consisting of triethoxysilane, trimethoxysilane, tributhoxysilane, sodium silicate, titanium isopropoxide, titanium butoxide, tin butoxide, and sodium stannate.

20. The method of claim 1, wherein the nanoparticles comprise a core-shell or multishell structure.
